(19) United States
(12) Patent Application Publication
(10) Pub. No.: US 2024/0423938 A1
(43) Pub. Date: Dec. 26, 2024

(54) FUNCTIONAL GUM COMPRISING AN ALGINATE BLEND

(71) Applicant: Reflux Gourmet LLC, Sacramento, CA (US)

(72) Inventor: Ken Frank, Napa, CA (US)

(73) Assignee: Reflux Gourmet LLC, Sacramento, CA (US)

(21) Appl. No.: 18/755,489

(22) Filed: Jun. 26, 2024

Related U.S. Application Data
(60) Provisional application No. 63/523,246, filed on Jun. 26, 2023.

(51) Int. Cl. A61K 31/197; A23G 4/06; A23G 4/08; A23G 4/10; A23G 4/12; A61K 9/00; A61K 33/00; A61K 36/886; A61K 36/9068; A61K 47/36

(52) U.S. Cl. CPC A61K 31/197; A23G 4/064; A23G 4/068; A23G 4/08; A23G 4/10; A23G 4/126; A61K 9/0065; A61K 33/00; A61K 36/886; A61K 36/9068; A61K 47/36

(57) ABSTRACT

The present invention relates to a composition for forming a protective barrier in the stomach, aimed at alleviating gastroesophageal reflux disease (GERD) and promoting oral and gastrointestinal health. The composition comprises an alginate complex including Sodium Bicarbonate, Sodium Alginate, Calcium Pantothenate, Ginger (root) Powder, Celery (root) Powder, and Aloe Vera (inner leaf) Powder. The composition forms a gel-like substance upon contact with saliva, creating a physical barrier that minimizes acid reflux. Additionally, the composition is incorporated into a chewing gum, which provides ease of use for the user and promotes overall oral hygiene. The gum includes natural sweeteners for preservation and improved taste, ensuring consumer acceptability.

### PRIORITY

The present application claims priority from the U.S. provisional application Ser. No. 63/523,246, which was filed on Jun. 26, 2023. The disclosure of that provisional application is incorporated herein as if set out in full.

### BACKGROUND OF THE DISCLOSURE

### Technical Field of the Disclosure

The present disclosure relates to gums, and specifically functional gums featuring an alginate blend.

### Background and Description of the Related Art

Functional food refers to food products that have additional health benefits beyond basic nutrition. Chewing gum is one such product that can be formulated to deliver functional ingredients.

Functional chewing gum typically contains added substances or ingredients that offer specific health benefits. These ingredients can vary but conventionally include vitamins, minerals, herbal extracts, dietary fibers, antioxidants, probiotics, prebiotics, amino acids, or other bioactive compounds. The gum acts as a delivery system, allowing the functional ingredients to be released and absorbed by the body through the oral cavity.

Functional chewing gums are generally designed to provide various health benefits, such as freshening breath, promoting oral health, improving digestion, enhancing cognitive function, boosting energy levels, supporting weight management, or delivering specific nutrients. For example, some functional gums may contain calcium and vitamin D for promoting bone health, or green tea extract for its antioxidant properties.

Formulation of functional chewing gum generally involves considerations such as the stability and bioavailability of the functional ingredients, taste, texture, and shelf life of the gum. The ingredients must be carefully selected and incorporated into the gum base while maintaining their efficacy.

Ideally, such gums are of desirable taste/odor despite their long-lasting acid-inhibiting effects, may improve the microbiology of the mouth or gut, may form a raft in vitro, and are optimized for useful dosage and delivery characteristics.

Chewing gum serves as an effective delivery system for functional ingredients. The act of chewing promotes salivation, which helps in releasing and distributing the bioactive compounds present in the gum. Many ingredients in such chewing gums are then absorbed by the body through the oral mucosa, providing systemic or localized benefits.

Such solutions typically act upon the stomach and esophagus. Following oral intake, food remains in the stomach between one and six hours. During that time, masticated food becomes more like a thin paste or gruel and is leaked in small portions to the duodenum. In order to process this food, several enzymes are produced by specialized epithelial cells. Furthermore, parietal cells produce hydrochloric acid, also called stomach acid, which is responsible for the low pH in the stomach. This stomach acid is a first-line defense to foreign organisms, providing a sterilizing effect on bacteria, fungi, and other microorganisms. Although essential to digesting food, stomach acid regurgitating back to the esophagus and upper airway is a problem in the modern world. The esophagus and throat (pharynx and larynx) are not equipped to handle the low pH stomach contents, and injury can occur. While many are genetically and physiologically predisposed to reflux, lifestyle factors such as stress, overeating, consuming food and drink too quickly, or consuming alcohol or too many carbonated drinks can further contribute to the regurgitation of stomach contents into the esophagus and upper airway.

Indigestion can be defined broadly as dyspepsia or GERD (Gastroesophageal Reflux Disease). Dyspepsia is a general term for the pain or discomfort a person can feel in the stomach and under the ribs, usually after eating or drinking (i.e., alcoholic beverages or coffee), although similar symptoms can occur on an empty stomach. GERD, on the other hand, is a digestive disorder caused by relaxation of the ring of muscle between one's esophagus and stomach. This ring is called the lower esophageal sphincter (LES), and the relaxation of it can further contribute to heartburn or acid indigestion.

Some people experience indigestion a few times per year while others suffer every day with symptoms ranging from mild discomfort lasting a few minutes to longer-lasting severe pain, sometimes accompanied by nausea and vomiting, which can go on for several hours. The most common indigestion symptoms are: pain or discomfort in the stomach and under the ribs, heartburn, feeling of being bloated or uncomfortably full after eating, rumbling or gurgling noises in the stomach, stomach cramps, a clenched or knotted feeling in the stomach, excessive burping or flatulence, and nausea or vomiting. The most common symptoms of regurgitation of gastric contents into the upper airway, or laryngopharyngeal reflux (LPR), are throat pain, throat clearing, hoarseness, cough, and the sensation of a lump in the throat (globus).

The disorder is experienced by an increasing number of people in the developed world. At least one third of the population suffers from episodic dyspepsia. Dyspepsia may be relieved by antacids, which work as bases or acid-buffering agents to raise the pH of the stomach contents. Antacids are normally over-the-counter products, which can be purchased at any pharmacy. Calcium carbonate is commonly used as the active ingredient in antacids. Some calcium-based antacids add other ingredients, such as magnesium or aluminum. Common over-the-counter antacids include Tums® sold by GlaxoSmithKline of Brentford, England, which is simply calcium carbonate; Rennie® sold by Bayer, which has magnesium added to ease the potential side effect of constipation with too much calcium; and Maalox®, sold by Novartis, which has aluminum added to calcium carbonate and comes as liquid or tablet forms. Antacids may be useful as self-medication in dyspeptic patients with mild or moderate heartburn, however, while they are fast-acting they are also short-acting, so they are less useful for frequent or severe heartburn and do not work well as a preventive measure.

Acid blockers such as H2 blockers and proton pump inhibitors (PPIs) are generally used for severe and chronic symptoms. These drugs work by blocking how much stomach acid is being produced. These acid blockers are not as fast-acting as antacids, but last longer and can be effective for several hours at a time. Over-the-counter H2 blockers include Axid®, Pepcid®, Tagamet®, and Zantac®. These brands are also available in prescription strength if the milder forms do not bring enough relief. The clinical efficacy of H2 blockers and proton pump inhibitors is well documented.

Medications such as Pepcid®, Tagamet®, and Zantac® work by blocking a type of histamine produced by the stomach, which in turn blocks acid production. These histamine blockers are typically taken twice a day, 30 to 60 minutes before eating to be most effective. Unfortunately, some histamine blockers have been implicated in the development of certain cancers, and many other serious, life-threatening, and/or life-altering side effects.

Another class of acid blocking drugs called proton pump inhibitors, or PPIs, shut down proton pumps in the stomach that produce acid, lowering acid levels dramatically. They are often used when histamine blockers do not provide enough relief or when people have erosions in the esophagus or other complications from GERD (Gastro Esophageal Reflux Disease). One proton pump inhibitor, Prilosec®, is available over the counter in the US as well as numerous other countries. Other PPIs, such as Aciphex®, Nexium®, Prevacid®, Protonix®, and other forms of Prilosec® may require a doctor's prescription depending on the regional requirements. Reglan® is another prescription drug that works to reduce acid reflux by speeding up how quickly the stomach empties. Reglan® strengthens the digestive contractions that move food through one's esophagus, and with this faster digestion can be associated with reduced heartburn. Reglan® and other pro-kinetic drugs have potentially mortal side effects and received a “black box” warning from the Food and Drug Administration, the agency's strongest warning.

Alginates are all-natural derivatives of kelp (brown seaweed) that have been used safely as natural food additives for over a hundred years. Alginate is a common household ingredient that is used to thicken foods and make jellies. When consumed, alginates also coat the lining of the esophagus and stomach creating a protective demulcent coating and form a protective alginate raft that floats on top of stomach contents to provide relief against acid reflux when combined with bicarbonate in the stomach. One of the major challenges to developing natural alginate products to help provide reflux relief is that they can form a medium for bacterial and fungal growth and are very difficult to preserve. Because of this, the primary preservative used in contemporary alginate-based products is paraben. Paraben is a synthetic preservative, a known hormone disruptor that has been implicated in hair loss, allergic reactions, and in the development of numerous cancers. Thus, there is a need for a natural, effective preservative to maintain the stability and safety of alginate-based products.

Recent studies have shown that natural preservatives such as grapefruit seed extract, rosemary extract, and essential oils (e.g., tea tree oil, thyme oil) can effectively inhibit the growth of bacteria and fungi in food products. These natural preservatives, when combined with alginates, can provide a safer alternative to synthetic preservatives, enhancing the shelf life and efficacy of functional chewing gums.

Furthermore, the incorporation of natural flavoring agents such as mint, fruit extracts, or spices can improve the taste profile of functional gums, making them more palatable to consumers. The challenge lies in balancing the flavors with the functional ingredients without compromising their bioavailability and effectiveness.

Another consideration in the formulation of functional gums is the texture. The gum base must be pliable and maintain its chewiness over time. Ingredients such as glycerin, lecithin, and various emulsifiers can be used to achieve the desired texture. These components also play a role in the controlled release of active ingredients, ensuring a consistent delivery of benefits throughout the chewing process.

### SUMMARY OF THE INVENTION

To minimize the limitations found in the prior art, and to minimize other limitations that will be apparent upon the reading of the specification, the preferred embodiment of the present invention provides a gum-based composition for mitigating the effects of stomach acid in a subject, said composition comprising an alginate complex and preferably one or more of Sodium Bicarbonate, Calcium Pantothenate, Sodium Alginate, Celery Powder, Ginger (root) Powder, Aloe Vera (inner leaf) Powder.

In other embodiments, the composition includes grapefruit seed extract, cofactors, excipients, and combinations thereof. In some embodiments, the composition further comprises at least one flavoring agent to enhance palatability.

It is a first objective of the present invention to provide a gum formulated as described herein, with a focus on the stability and bioavailability of the functional ingredients, sensory attributes like taste and texture, and shelf life of the gum while meeting regulatory requirements.

A second objective is to provide a gum that has a demulcent effect, which also cleanses the user's teeth with bicarbonate. The demulcent effect helps protect the mucosal lining of the throat and esophagus from acid reflux.

Another objective of the present invention is to provide the above benefits but also with the presence of ginger.

Another objective of the present invention is to provide the above benefits but also with the presence of aloe.

It is the object of the present invention to disclose a nutritional aid/supplement (or “digestion aids”) containing alginates that are optimized for improved solubility, safety, taste and effectiveness. Said digestion aids benefit from nearly instantaneous action as well as longer lasting neutralizing effects on stomach acid, in contrast to the prior art described above. Thus, the herein described invention satisfies a need in art for safer, palatable, non-hormone disrupting, non-carcinogenic digestive aids, and in addition is all-natural, paraben-free, and safe.

These and other advantages and features of the present invention are described with specificity so as to make the present invention understandable to one of ordinary skill in the art.

### DETAILED DESCRIPTION OF THE DRAWINGS

In the following discussion that addresses a number of embodiments and applications of the present invention, reference is made to the accompanying drawings that form a part hereof, and in which are shown by way of illustration specific embodiments in which the invention may be practiced. It is to be understood that other embodiments may be utilized, and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. As used herein, the term ‘about” means+/−5% of the recited parameter. All embodiments of any aspect of the invention can be used in combination unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; said again may refer to language indicating “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a preferred embodiment, the composition comprises an alginate complex of 50 mg of Alginate Complex (Sodium Bicarbonate, Calcium Pantothenate, Sodium Alginate, Celery Powder, Ginger (root) Powder, Aloe Vera (inner leaf) Powder). Table 1, below, shows an exemplary embodiment:

TABLE 1

| Ingredient | Serving | |
|---|---|---|
| Description | (mg) | % Weight |
| Gum Base | 525 | ~26 |
| Sorbitol | 425 | ~21 |
| Maltitol | 320 | ~16 |
| Xylitol | 275 | ~14 |
| Isomalt | 210 | ~10 |
| Sodium | 45 | ~2.1 |
| Bicarbonate | | |
| Sodium | 2.4 | ~0.12 |
| Alginate | | |
| Calcium | 2.4 | ~0.12 |
| Pantothenate | | |
| Ginger (root) | 2.4 | ~0.12 |
| Powder | | |
| Celery (root) | 2.4 | ~0.12 |
| Powder | | |
| Aloe Vera | 2.4 | ~0.12 |
| (inner leaf) | | |
| Powder | | |
| Natural Flavor | 80 | ~4.0 |
| Citric Acid | 5 | ~0.25 |
| (Flavor) | | |
| Malic Acid | 11 | ~0.60 |
| (Flavor) | | |
| Magnesium | ~60 | ~3.1 |
| Stearate | | |
| Silicon | 8 | ~0.40 |
| Dioxide | | |
| Stevia | ~13 | ~0.67 |
| Glycerin | 6.4 | ~0.32 |
| Total | ~2,000 | ~100 |

In various embodiments of the composition, the amounts of the specified chemical components can vary within certain ranges to achieve desired properties and effects. In a highly preferred embodiment, the composition comprises Sodium Bicarbonate in the amount of 2% by weight, Sodium Alginate in the amount of 0.1% by weight, Calcium Pantothenate in the amount of 0.1% by weight, Ginger (root) Powder in the amount of 0.1% by weight, Celery (root) Powder in the amount of 0.1% by weight, and Aloe Vera (Inner leaf) Powder in the amount of 0.1% by weight.

In a somewhat less preferred embodiment, the ranges of the components are slightly broader. Here, Sodium Bicarbonate may range from 1.5% to 2.5% by weight, Sodium Alginate from 0.08% to 0.12% by weight, Calcium Pantothenate from 0.08% to 0.12% by weight, Ginger (root) Powder from 0.08% to 0.12% by weight, Celery (root) Powder from 0.08% to 0.12% by weight, and Aloe Vera (Inner leaf) Powder from 0.08% to 0.12% by weight.

A broader range is considered in another embodiment, where Sodium Bicarbonate is present in amounts ranging from 1% to 3% by weight, Sodium Alginate from 0.05% to 0.2% by weight, Calcium Pantothenate from 0.05% to 0.2% by weight, Ginger (root) Powder from 0.05% to 0.2% by weight, Celery (root) Powder from 0.05% to 0.2% by weight, and Aloe Vera (Inner leaf) Powder from 0.05% to 0.2% by weight.

In even less preferred embodiments, the ranges are expanded further to accommodate more significant variations. For example, Sodium Bicarbonate can range from 1% to 4% by weight, Sodium Alginate from 0.05% to 0.25% by weight, Calcium Pantothenate from 0.05% to 0.25% by weight, Ginger (root) Powder from 0.05% to 0.25% by weight, Celery (root) Powder from 0.05% to 0.25% by weight, and Aloe Vera (Inner leaf) Powder from 0.05% to 0.25% by weight.

In the broadest embodiment, the ranges for the chemical components are maximized to ensure the composition can be adapted for a wide array of purposes. Sodium Bicarbonate may be included in amounts ranging from 0.5% to 5% by weight, Sodium Alginate from 0.05% to 0.3% by weight, Calcium Pantothenate from 0.05% to 0.3% by weight, Ginger (root) Powder from 0.05% to 0.3% by weight, Celery (root) Powder from 0.05% to 0.3% by weight, and Aloe Vera (Inner leaf) Powder from 0.05% to 0.3% by weight.

In some embodiments, the range of the components may not have an upper bound, providing significant flexibility for increasing the concentration of each ingredient as needed. For instance, Sodium Bicarbonate could be present in amounts starting from 1% by weight with no upper limit, allowing for higher concentrations based on specific application requirements. Similarly, Sodium Alginate, Calcium Pantothenate, Ginger (root) Powder, Celery (root) Powder, and Aloe Vera (Inner leaf) Powder could each begin from 0.05% by weight, with no specified maximum. This approach enables the formulation to be tailored for enhanced efficacy or potency, depending on the desired characteristics of the final product.

Conversely, some embodiments may specify ranges without a lower bound, ensuring the inclusion of components up to a certain maximum concentration while allowing for extremely low or even negligible amounts. For example, Sodium Bicarbonate could be included up to 300 by weight without a lower limit, accommodating formulations where minimal quantities are sufficient. Sodium Alginate, Calcium Pantothenate, Ginger (root) Powder, Celery (root) Powder, and Aloe Vera (Inner leaf) Powder could each be present up to 0.500 by weight, with no minimum specified. This flexibility is beneficial for formulations where the presence of trace amounts of certain components is adequate to achieve the desired effect, or where manufacturing constraints necessitate lower ingredient usage.

Additional details may be found in the following table:

TABLE 2

| Serving Size (mg) | 2,000 | | |
|---|---|---|---|
| Appearance | Round Tablet | | |
| Color | Uneven off-white with | | |
| | specs | | |
| Odor | Berry | | |
| Taste | Berry with celery & | | |
| | ginger | | |
| Servings per container | 30 | | |
| Calories | 0 | | |
| Sodium | 15 | mg | 1% |
| Total Carbohydrates | 1 | g | <1% |
| Total Sugars | 0 | g | ** |
| Added Sugars | 0 | g | 0% |

Reflux Gourmet's Propriety Alginate Complex
(Sodium Bicarbonate, Sodium Alginate, Calcium
Pantothenate, Ginger (root) Powder, Celery
Powder, Aloe Vera
(leaf) Powder)

| | |
|---|---|
| Composition | 50 mg |
| Other Ingredients | Gum base, sorbitol, maltitol, xylitol, isomalt, |
| | natural flavors, magnesium stearate, stevia, |
| | silicon dioxide, glycerin |
| Composition | Gum base, sorbitol, maltitol, xylitol, isomalt, |
| | natural flavors |
| | (Flavors, malic acid, citric acid), magnesium |
| | stearate, stevia, silicon dioxide, glycerin |
| Allergen Statement | Allergen Free (All common allergens) |
| Gluten | Gluten Free |
| Sugar Content | Sugar Free (Sugar alcohol) |
| Flavor | Natural Flavors |
| Shelf Life | 24 months |

In some embodiments, the ratio of Sodium Bicarbonate to the other named ingredients Sodium Alginate, Calcium Pantothenate, Ginger (root) Powder, Celery (root) Powder, Aloe Vera (inner leaf) Powder is 20:1 as shown above. In still other embodiments, it is 5:1, 10:1, 15:1, 30:1, 40:1, or 50:1. In other embodiments, it ranges between any of the above ratios, and in still other embodiments, some of the Calcium Pantothenate, Ginger (root) Powder, Celery (root) Powder, Aloe Vera (inner leaf) Powder ingredients are in one of the above ratios or range between the above ratios while other ingredients are another of the above ratios or range between the above ratios.

In still other embodiments, the ratios vary to accommodate different formulation needs and desired outcomes. Ratios such as 5:1, 10:1, 15:1, 30:1, 40:1, or 50:1 are considered. For example, a 5:1 ratio may be utilized when a more balanced effect from both Sodium Bicarbonate and the other ingredients is desired, whereas a 50:1 ratio may be optimal for applications requiring an even higher concentration of Sodium Bicarbonate relative to the other components. These variations allow for fine-tuning the formulation to meet specific performance criteria, ensuring that the resulting product is tailored to the intended use.

Additionally, in some embodiments, the ratios can range between any of the specified ratios, providing a spectrum of possibilities. For instance, the ratio might range from 10:1 to 30:1, offering flexibility within that interval to achieve a balanced or targeted effect. This approach allows manufacturers to adjust the ratios dynamically based on the specific requirements of different batches or product lines, ensuring consistent quality and efficacy while accommodating variations in raw material properties or processing conditions.

In further embodiments, the ratios of some ingredients might differ from others, providing an even greater degree of customization. For instance, while Calcium Pantothenate and Ginger (root) Powder might be maintained at a 10:1 ratio with Sodium Bicarbonate, Celery (root) Powder and Aloe Vera (inner leaf) Powder could be at a 30:1 ratio.

Alginates function to alleviate GERD by forming a protective barrier on top of the stomach contents, helping to minimize stomach acid from flowing back into the esophagus. When the gum is chewed, the alginate ingredient mixes with saliva and forms a gel-like substance. This gel floats on the stomach contents and forms a physical barrier, reducing the likelihood of acid reflux.

The primary reason sodium bicarbonate is not normally used with functional chewing gums is that sodium bicarbonate has a salty taste, which may not be desirable in chewing gum products. Additionally, sodium bicarbonate can lose its effectiveness when exposed to air and moisture, which could affect the shelf life and efficacy of chewing gum formulations.

The term ‘alginate’ is intended to encompass alginic acids, salts of alginic acids (alginate salts), derivatives of alginic acid, for example esters such as propylene glycol and mixtures thereof.

In some embodiments, dextrose sugars, and/or other all-natural free sweeteners contribute to the desiccation of the product which facilitates its action as a preservative. In addition, in some embodiments, the composition forms a demulcent (protective) barrier on the mucosal lining of the throat and esophagus of a user to provide relief against the partially regurgitated low pH constituents of the small intestine and stomach characteristic of gastroesophageal reflux.

In use, the act of gum chewing causes the release of saliva, which is the body's endogenous antacid. Chewing sodium bicarbonate may be useful in the minimization of the formation of cavities and for cleaning the teeth. Gum chewing also promotes increased swallowing frequency which helps clear any refluxed stomach contents. Adding the proposed invention with alginate formula adds a protective coating (demulcent) to the throat and esophagus.

The gum is preferably of desirable taste/odor despite its long-lasting acid-inhibiting effects, improves the microbiology of the mouth, large intestine, and small intestine, forms a raft in vitro—also tested in vitro models of the mouth containing salivary amylase, peptidases, and the like, and is optimized for useful dosage and delivery characteristics.

In a preferred method of manufacture, the following ingredients are blended: Sodium Bicarbonate, Sodium Alginate, Calcium Pantothenate, Ginger (root) Powder, Celery Powder, Aloe Vera (leaf) Powder (approx. 50 mg), gumbase, sorbitol, isomalt, xylitol, maltitol, magnesium stearate, natural flavors, stevia extract, silicon dioxide, and confection glaze. The resultant gum powder is stamped in a tablet press to form individual pieces of gum.

Next, the gum pieces are loaded into conventional panning equipment. One or more layers of polyol syrup are applied using a sprayer or ladle. In this embodiment, the polyol syrup may comprise several sugar alcohols including but not limited to maltitol, xylitol, and isomalt. The rotational motion of the pan facilitates the spreading and subsequent drying of the syrup, forming a crunchy shell that covers the gum pieces. The gum pieces are then held for a minimum of 12 hours before packaging. Optionally, the syrup is made using mainly sugar alcohols and other ingredients for taste and/or color. This may include panning using maltitol, xylitol, isomalt or any other “sugar-free” (specifically, free of sucrose) ingredients. Optionally, other customized flavoring and/or coloring systems may be used.

### Additional Embodiments and Applications

In one embodiment, the functional gum includes a combination of alginate with other gelling agents such as pectin or agar to enhance the protective barrier effect. This combination can provide a stronger and more resilient barrier against stomach acid, improving the overall efficacy of the product in managing GERD symptoms.

Another embodiment focuses on optimizing the release profile of active ingredients. By incorporating microencapsulation techniques, the active ingredients can be protected from degradation and released gradually over time, ensuring prolonged therapeutic effects. This approach can be particularly beneficial for ingredients like ginger and aloe vera, which can lose their potency when exposed to the environment.

The use of advanced flavor masking technologies can also be incorporated to improve the palatability of the gum. For instance, encapsulating the sodium bicarbonate in a lipid-based coating can mask its salty taste, making the gum more enjoyable to chew without compromising its functional properties.

To enhance the shelf life and stability of the functional gum, natural antioxidants such as vitamin E and rosemary extract may be added. These ingredients can help prevent the oxidation of sensitive compounds, ensuring the gum retains its efficacy and freshness over an extended period.

Another innovative application involves the use of probiotics in the gum formulation. By incorporating specific strains of beneficial bacteria, the gum can further promote gut health and support the immune system.

The functional gum may also be designed to address specific dietary requirements. For example, a sugar-free formulation using natural sweeteners like stevia and monk fruit extract can be developed for individuals managing diabetes or those following a low-sugar diet. Similarly, a gluten-free formulation can cater to individuals with celiac disease or gluten intolerance.

The development of biodegradable gum bases is another area of interest. Traditional gum bases are often synthetic and non-biodegradable, contributing to environmental pollution. By exploring natural and biodegradable alternatives, such as chicle or other plant-based materials, the functional gum can offer a more sustainable option for environmentally conscious consumers.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1) A gum comprising Sodium Bicarbonate, Calcium Pantothenate, Sodium Alginate, and Celery Powder.

2) The gum according to claim 1, further comprising ginger.

3) The gum according to claim 1, further comprising aloe.

4) The gum according to claim 1, wherein the gum is formulated to release the active ingredients over a prolonged period.

5) The gum according to claim 1, wherein the gum base comprises a blend of natural and synthetic polymers.

6) A composition for forming a protective barrier in the stomach, comprising:
a) Sodium Bicarbonate in an amount of 1.5% to 2.5% by weight;
b) Sodium Alginate in an amount of 0.08% to 0.12% by weight;
c) Calcium Pantothenate in an amount of 0.08% to 0.12% by weight;
d) Ginger (root) Powder in an amount of 0.08% to 0.12% by weight;
e) Celery (root) Powder in an amount of 0.08% to 0.12% by weight; and
f) Aloe Vera (inner leaf) Powder in an amount of 0.08% to 0.12% by weight.

7) The composition of claim 6, wherein the ratio of Sodium Bicarbonate to the other ingredients is 20:1.

8) The composition of claim 6, wherein the ratio of Sodium Bicarbonate to the other ingredients is 10:1.

9) The composition of claim 6, wherein the ratio of Sodium Bicarbonate to the other ingredients is 5:1.

10) The composition of claim 6, further comprising dextrose sugars as a natural sweetener.

11) A method for alleviating gastroesophageal reflux disease (GERD) in a subject, comprising:
a) Administering to the subject a composition comprising:
i) Sodium Bicarbonate in an amount of 1% to 3% by weight;
ii) Sodium Alginate in an amount of 0.05% to 0.2% by weight;
iii) Calcium Pantothenate in an amount of 0.05% to 0.2% by weight;
iv) Ginger (root) Powder in an amount of 0.05% to 0.2% by weight;
v) Celery (root) Powder in an amount of 0.05% to 0.2% by weight; and
vi) Aloe Vera (inner leaf) Powder in an amount of 0.05% to 0.2% by weight;
b) Wherein the composition forms a gel-like substance upon contact with saliva to create a physical barrier in the stomach.

12) The method of claim 11, wherein the ratio of Sodium Bicarbonate to the other ingredients ranges between 5:1 and 50:1.

13) The method of claim 11, wherein the gel-like substance forms a raft on top of the stomach contents.

14) The method of claim 11, wherein the composition includes propylene glycol alginate as the alginate component.

15) A chewing gum composition for promoting oral and gastrointestinal health, comprising:
a) An alginate complex including:
i) 50 mg of Alginate Complex containing:
(1) Sodium Bicarbonate;
(2) Calcium Pantothenate;
(3) Sodium Alginate;
(4) Celery Powder;
(5) Ginger (root) Powder; and
(6) Aloe Vera (inner leaf) Powder;
ii) Dextrose sugars and/or other all-natural free sweeteners to facilitate desiccation and preservation; and
b) Wherein the gum forms a demulcent barrier on the mucosal lining of the throat and esophagus when chewed.

16) The chewing gum composition of claim 15, wherein the ratio of Sodium Bicarbonate to the other ingredients is 30:1.

17) The chewing gum composition of claim 15, wherein the ratio of Sodium Bicarbonate to Calcium Pantothenate and Ginger (root) Powder is 10:1, and the ratio to Celery (root) Powder and Aloe Vera (inner leaf) Powder is 30:1.

18) The chewing gum composition of claim 15, wherein the chewing gum is optimized for controlled release of the active components.

19) The chewing gum composition of claim 15, wherein the gum base is derived from natural chicle.

20) The chewing gum composition of claim 15, further comprising xylitol as a sugar substitute.
